(19) United States
(12) Patent Application Publication
(10) Pub. No.: US 2005/0039743 A1
(43) Pub. Date: Feb. 24, 2005

(54) Medicament dispenser

(76) Inventor: Anthony James Taylor, Ware (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/499,222

(22) PCT Filed: Dec. 20, 2002
(86) PCT No.: PCT/EP02/14827

(30) Foreign Application Priority Data
Dec. 22, 2001 (GB) 0130857.6

(51) Int. Cl. A61M 15/00; A61M 11/00; A61M 16/00

(52) U.S. Cl. 128/203.15; 128/200.23

(57) ABSTRACT

There is provided a medicament container or delivery device for use with medicament in dry powder form comprising a material that reduces the surface energy or increases the electrical conductivity of some or all parts of the medicament container or delivery device which come into contact with said dry powder form medicament. Suitably, the material has friction-reducing or electrical conductivity-enhancing properties.

### TECHNICAL FIELD

The present invention relates to containers and dispensers for medicament powders. In particular, the invention relates to dry powder inhalation dispensers and components thereof which substantially alleviate or reduce the undesirable adherence of medicament thereto.

### BACKGROUND OF THE INVENTION

Medicaments for treating respiratory disorders are frequently administered as dry powder formulations through the mouth and nose. Dry powder medicament dispensers, such as inhalers, are used in the administration of these drugs, inhalation by the patient resulting in uptake of a specified dosage of medicament through the nose or mouth. The drug may be stored as a dry powder within a reservoir in the body of the inhaler, a metering chamber being utilised to administer a specified dose of medicament. Alternatively, more sophisticated medicament dispensers employ medicament carriers, such as individual capsules or blister packs/strips containing defined doses of powdered drug.

Patients often rely on medication delivered by dry powder inhalers for rapid treatment of respiratory disorders that are debilitating and in some cases life threatening. It is, therefore, essential that the prescribed dose of drug is delivered accurately and consistently to meet the patient's needs and comply with the requirements of regulatory authorities.

A problem which can occur in the storage and product lifetime of an inhaler is the undesirable adherence (e.g. sticking) of the medicament powder to the internal surfaces thereof. The tendency for medicament to so adhere can potentially affect the administration of an effective dose of medicament by reducing the amount of free (i.e. non-adhered) medicament available for uptake (e.g. by inhalation) by the patient.

The Applicants have found that the above described problem may be ameliorated by coating any of the parts of the medicament container or delivery apparatus/device which contact the medicament with materials which reduce the surface energy or increase the electrical conductivity thereof. Use of said coatings on can also improve the flow of medicament powder through the delivery apparatus/device.

The Applicants have also found that further amelioration of the above-described problems is achievable by controlling the ingress of moisture to, or egress of moisture from, the medicament container. Control may be achieved by either suitable choice of container materials or by enclosure of the container or a dispenser including the container in a suitable package. The control need not absolutely prevent moisture transfer. Indeed, the Applicants have found that under certain conditions a limited degree of moisture transfer can be desirable.

### SUMMARY OF INVENTION

According to the present invention, there is provided a medicament container or delivery apparatus/device for use with medicament in dry powder form comprising a coating that reduces the surface energy or increases the electrical conductivity thereof.

In alternative aspects herein, the coating is substituted by the incorporation (e.g. impregnation) of suitable surface energy reducing or electrical conductivity enhancing materials into the bulk of any or all of the parts that make up the medicament container or delivery apparatus/device.

In a first aspect, the coating acts such as to reduce the surface energy of the parts of the medicament container or delivery apparatus/device that come into contact with the dry powder form medicament. Suitable coatings include any surface energy reducing material including frictional coatings such as those coatings comprising fluoropolymers.

Suitably, the surface energy of the coating gives a contact angle of greater than about 70 degrees, preferably greater than about 100 degrees, more preferably greater than about 110 degrees. As used herein, “contact angle” is the angle between a liquid water droplet and a solid surface of the modified surface/bulk at the liquid/solid interface.

Suitably, the coating thickness is in the range of about 1 nm to about 200 nm such as about 10 nm to about 100 nm.

In one aspect, the coating comprises a fluoropolymer. Suitable fluoropolymers include those comprising multiples of one or more of the following monomeric units: tetrafluoroethylene (PTFE), fluorinated ethylene propylene (FEP), perfluoroalkoxyalkane (PFA), ethylene tetrafluoroethylene (ETFE), vinyldienefluoride (PVDF), and chlorinated ethylene tetrafluoroethylene. Fluorinated polymers, which have a relatively high ratio of fluorine to carbon, such as perfluorocarbon polymers, e.g., PTFE, PFA and FEP are particularly suitable.

The fluoropolymer is optionally blended with a non-fluorinated polymer such as polyamides, polyimides, polyamide imides, polyethersulfones, polyphenylene sulfides, and amine-formaldehyde thermosetting resins. These added polymers often improve adhesion of the polymer coating to the substrate. Preferred polymer blends are PTFE/FEP/polyamideimide, PTFE/polyether sulphone (PES) and FEP-benzoguanamine. The most preferred polymer coating is a blend of PTFE and PES. A coating of pure FEP is also of considerable interest.

A technique for applying such coatings to for example, a metal blister, such as aluminium or stainless steel, is where the metal is precoated as coil stock and cured before being stamped or drawn into the appropriate shape. This method is well suited to high volume production for two reasons. First, the art of coating coil stock is well developed and several manufacturers can custom coat metal coil stock to high standards of uniformity and in a wide range of thicknesses. Second, the precoated stock can be stamped or drawn at high speeds and precision by essentially the same methods used to draw or stamp uncoated stock.

Other techniques for coating techniques includes electrostatic dry powder coating or by spraying preformed components with formulations of the coating and then curing. The components may also be dipped in the coating formulation and cured, thus becoming coated on the inside and out. The coating formulation may also be poured inside the components then drained out leaving the insides with the polymer coat.

The appropriate curing temperature is dependent on the particular coating and the coating method employed.

However, for coil coating and spray coating temperatures in excess of the melting point of the polymer are typically required, for example, about 50° C. above the melting point for up to about 20 minutes such as about 5 to 10 minutes e.g. about 8 minutes or as required. For the above named preferred and particularly preferred polymer blends curing temperatures in the range of about 300° C. to about 400° C., e.g. about 350° C. to 380° C. are suitable.

Where the components are coated and then cured the substrate components may be prepared from strengthened materials to ensure they withstand the process.

Conversely alternative polymer coatings may be used on the components which may be dipped or bath immersed into a treatment tank containing a solution of polymeric compound. Usually the components are immersed in the solution at room temperature for at least one hour, for example, 12 hours, thus being treated both internally and externally.

The treated components are preferably washed with solvent and dried at an elevated temperature for example 50-100° C. optionally under vacuum.

Further examples of suitable coating materials include of fluoropolyethers having functionalised ends groups with a general formula Rᵣ—O(C₃F₆O)ₘ(CFX)ₙ—CFX—Y-Zₚ as described in U.S. Pat. No. 4,746,550 (incorporated herein by reference) including perfluoropolyethers having functional groups capable of anchoring the coating to the substrate such as carboxyl, ester, amide, hydroxyl, isocyanate, epoxy, silane for example —CONR²R³ wherein R² and R³ may be independently selected from amongst other things hydrogen, or a silyl ether (e.g. SiRₜ(OR)₃₋ₜ or a fluoropolyether having hydroxylic functionality of the type —CF₂CH₂OH, —CF₂CFXCH₂OH (wherein X is Cl or F) or —CF(CF₃)CH₂OH as described in U.S. Pat. No. 6,071,564 (incorporated herein by reference); phosphoric diesters of formula [XCF₂CF₂O(CFXCF₂O)ₓCFXCH₂O]₂PO(OM) as described in U.S. Pat. No. 3,492,374 (incorporated herein by reference) or phosphoric monoester of formula [R_{f}—O—CFY-L-O]ₘP═O(O⁻Z⁺)₃₋ₘ as described in EP 0 687 533 (incorporated herein by reference) wherein L is a divalent organic group; m=1; Y is —F or —CF₃; Z⁺ is selected from H⁺, M⁺ where M is an alkali metal; N(R)₄⁺ where the R groups independently represent H or C₁₋₆alkyl; R_{f} is a polyperfluoroalkyleneoxide chain.

The fluoropolyethers described above may be used in combination with monofunctional fluoropolyethers having —CH₂OH terminals directly linked to a perfluoroalkyl group —CF₂, —CF₂CFX (wherein X is Cl or F) or CF(CF₃) optionally through a bridging group (CH₂CH₂)_{q} wherein q represents an integer from 1 to 6.

Other suitable coating materials also include polymeric compounds that are silane derivatives of perfluoropolyoxyalkanes with a molecular weight in the range 1600-1750 and those of the general formula:
R¹—(CH₂)ᵥ—CF₂O—(C₂F₄O)ₓ—(CF₂O)_{y}CF₂—(CH₂)_{w}—R¹ (I)
wherein R¹ comprises:
—(OCH₂—CH₂)_{z}—OPO(OH)₂, wherein x, y and z are such that the molecular weight of the compound is 900-2100 and v and w independently represent 1 or 2.

In one preferred aspect, the fluoropolymer coating results from surface treatment of the medicament container or delivery apparatus/device. Most preferably the surface treatment comprises a process of plasma coating e.g. with highly fluorinated small molecules such as: C₁₋₁₀perfluoroalkanes including fluorocycloalkanes; C₂₋₁₀perfluoroalkenes; fluoroalkanes including fluorocycloalkanes or fluoroalkenes wherein a high proportion of the hydrogens have been replaced by fluorines or mixtures thereof as described above.

In one aspect, the fluoropolymer plasma coating comprises a fluorine/carbon ratio of greater than about 1.4. Typically, the plasma coating comprises greater than about 45% CF₂ (PTFE) and CF₂ moieties. The number of CF₂ moieties can be determined using XPS.

Suitably, the fluorinated coating is prepared by the plasma polymerisation of a CₙF₂ₙ₊₂ monomer, wherein n is from 2 to 10. Suitably, n is from 3 to 8, preferably 6.

The plasma coating applied to one or more internal surfaces of the medicament container or delivery apparatus/device may be prepared from plasma generated substantially from CₙF₂ₙ₊₂ monomer. Alternatively, the plasma may be generated from a mixture of CₙF₂ₙ₊₂ monomer and one or more unsaturated monomers. The unsaturated monomer employed and the quantity thereof may be varied as part of optimisation of the employment of the invention. All combinations thereof are within the scope of the invention.

In one suitable method of plasma coating, polymerisation or modification of a hydrocarbon-containing pre-coating on the surface by interchange of hydrogen ions in the material with fluorine ions is employed. The plasma coating process typically occurs under vacuum. The components to be coated are placed inside a chamber, which is then evacuated. The CₙF₂ₙ₊₂ monomer (and optionally additional polymeric material) is introduced into the chamber at a controlled rate. The monomer gas is ignited within the chamber and maintained for a given time at a chosen power setting. For plasma polymerisation the chamber temperature is typically in the range of about 20° C. to about 150° C. At the end of the treatment the plasma is extinguished, the chamber flushed and the coated products retrieved. During the polymerisation process, a thin layer of plasma polymer will be bonded to the medicament container or delivery apparatus/device.

Accordingly, a further aspect of the invention provides a process for coating one or more of the internal surfaces of (e.g. a component of) the medicament container or delivery apparatus/device with a fluorinated coating, said process comprising the steps of (i) placing the component to be coated in a chamber, (ii) evacuating the chamber, (iii) feeding monomer of formula CₙF₂ₙ₊₂ monomer, wherein n is from 2 to 10 into the chamber, (iv) applying sufficient power to generate a plasma, (v) igniting the plasma, (vi) extinguishing any unreacted plasma, and (vii) flushing the chamber.

In a further aspect, step (iii) further includes the additional of one or more additional unsaturated monomers into the chamber with the CₙF₂ₙ₊₂ monomer.

The effectiveness of the fluorinated coating as hereinbefore defined depends on the operating conditions under which the plasma reactor operates. The operating parameters, which can be varied, include: power (W), gas pressure (mTorr), gas flow (cc/min), tumbler speed (rpm) and temperature (° C.).

The positioning and/or orientation of the components within the reactor may affect the effectiveness of the coating. It is believed that the components to be coated should be positioned within the primary plasma in the reactor. In order to obtain a uniform coating on all the components, the components should be evenly distributed in the reactor.

Suitably, to improve adhesion of the fluorinated coating to the internal surfaces, the surfaces to be coated are subjected to a pre-treatment step to remove any surface contamination and/or to activate the surface. The component surfaces to be coated may be pre-treated with an etching gas such as oxygen or argon. Preferably, the etching gas is oxygen. In the process, radicals react with the plastic or metal substrate; for example the component is exposed to a low pressure oxygen plasma environment which creates polar groups on the component's surface which are more conducive to bonding with the plasma coating to be applied.

The pre-treatment step, for example with oxygen, could be carried out under a range of conditions and duration. The pre-treatment process is dependent on the material to be treated.

Alternatively, the medicament container or delivery apparatus/device (especially when composed of a plastics material for example those described above) may be surface treated with a siloxane such as dimethyl siloxane using a similar process as that described above for fluoropolymer plasma coating.

In another aspect, the fluoropolymer coating is prepared by a process comprising polymerisation of fluorine-containing radicals on one or more surfaces of the medicament container or delivery apparatus/device. Polymerisation of the radicals may occur subsequent to generation of the radicals, or polymerisation may begin while additional radicals are being generated.

The radicals for use in such a process may be generated by a number of processes including, but not limited to, a microwave plasma process, a pulsed wave plasma process, hot filament chemical vapour process, pyrolisation of fluoroparylene dimers, using a photo initiator to create radicals from a fluoroacrylate and laser ablation of a fluoropolymer target.

A suitable microwave plasma process is carried out in a continuous or a pulsed manner at an energy range of 300 MHz to 300 GHz, for example at energy of 2.45 GHz. A microwave process can be more efficient than a conventional continuous RF process and consequently the quantity of monomer gas required to provide equivalent coating may be less. Furthermore, the coating times required are of the order of seconds as opposed to minutes or even hours for continuous RF triggered plasma process. Accordingly, any potential damage to the substrate can be reduced. Non-fluorinated monomers can be used in addition to fluorinated monomers, the ratio of non-fluorinated to fluorinated either being constant throughout the process or varying. Suitably, the ratio is high at the beginning of the process and low at the end. Preferably, pure non-fluorinated monomer is used at the beginning of the process and pure fluorinated monomer used at the end of the process.

A pulsed wave plasma process provides potential advantages over the traditionally used continuous RF plasma processes. The pulsed process is more controllable since the average energy is lower. This results in less damage to the substrate and more control over the radical polymerisation. Pulses are applied on and off at intermittent intervals, suitably the ‘off’ time being longer than the ‘on’ time. Suitably, the ratio of ‘off’ time to ‘on’ time is such that the average power applied is less than 10 Watts, preferably less than 1 W. For example, the power may be applied for 20 μS, followed by no power for 10000 μS to 20000 μS. Furthermore, the ‘off’ time allows the radicals to penetrate into intricate substrates providing an improved coating for more intricate substrates. The energy applied may be in the microwave or RF wave range.

In the case of a hot filament chemical vapour process, a fluorinated compound, such as a fluorinated gas, is heated to provide fluorine-containing radical, such as CF₂ radicals, which subsequently condense and polymerise on the surface of a substrate to form an essentially PTFE coating. This process is a simple process that can be used to coat intricate substrates and which causes no damage to the substrate.

Alternatively, fluoroparylene dimmers can be heated and then pyrolised to form a di-radical monomer, which condenses and polymerises on a cooled substrate. This is a simple process, which can be used to coat intricate substrates and causes no damage to the substrate. Furthermore, the composition of the coating will be known.

Alternatively, a photo initiator, for example of formula PhCh₂C(OH)(CH₃)CHO, is used to create radicals from a fluoroacrylate, for example of formula C₈F₁₇CH₂CH₂OCO═CH₂. The radicals so formed then polymerise on one or more of the surfaces of the medicament container or delivery apparatus/device.

Alternatively, fluorinated radicals may be obtained by laser ablation of a fluoropolymer target. For example, a pulsed laser may be directed onto sintered fluoropolymer, such as PTFE, in an argon atmosphere, the fluoropolymer volatilises to form fluorine-containing radicals, the radicals polymerising on one or more surfaces of the medicament container or delivery apparatus/device.

In another aspect, the coating acts such as to enhance the electrical conductivity of the parts of the medicament container or delivery apparatus/device which come into contact with the dry powder form medicament.

Suitable conductive coatings include those comprising a ceramic material. Suitably, the ceramic material comprises a cermet material. More suitably, the cermet material comprises a composite of a ceramic material with a conductive metal, or a ceramic material doped with a conductive metal. The cermet material is selected from the group consisting of nickel zirconate, cobalt zirconate and molybdenum zirconate.

Other suitable conductive coatings comprise an organic polymeric material. Suitably the organic polymeric material is selected from the group consisting of polycarbonate, polyester, acetal, nylon and epoxy resins. More suitably the organic polymeric material includes a doping element selected from the group consisting of gold, silver and similar precious metals, carbon black and quaternary ammonium salts. The doping elements are taken to include elements which are sputtered, loaded or doped onto a target. Targets can comprise polymeric materials such as polycarbonate, polyester, acetal, nylon or epoxy resins.

In one aspect, the container is suitable for containing a measured dose of medicament. Packs in blister pack form for the containment of a unit dose medicaments are envisaged, as are packs containing multiple unit dose blisters arranged sequentially or otherwise, such as in series form. A particular multi-unit dose arrangement comprises an elongate strip having multiple blisters arranged in series thereon.

In another aspect, the container is a reservoir for dry powder medicament. Metering means are provided to enable metering of dose from the reservoir and transport of that dose to a delivery position.

In one aspect, the container is a medicament dispenser comprising a body defining a reservoir for medicament in powder form, and an outlet in communication with said reservoir for release of the medicament. In one aspect, the device is an inhaler and the outlet is one through which a user can inhale.

In another aspect, the container is in the form of a reloadable cartridge comprising a medicament pack (e.g. in multi-unit dose blister form or reservoir form). The cartridge is shaped and sized for receipt by a medicament delivery device (e.g. an inhaler device).

In another aspect, the container is a medicament dispenser comprising a body defining a chamber for receipt of a medicament carrier, and an outlet in communication with said chamber for release of the medicament. In one aspect, the device is an inhaler and the outlet is one through which the user can inhale.

In one aspect, the coating material coats the body e.g. part or whole of the inside of the body. In another aspect, that material is impregnated throughout the body.

In another aspect, the coating material lines the body (e.g. the interior of the body which contacts the medicament powder in use).

In another aspect, the coating material is located around a seal for sealing the reservoir or medicament carrier. In a particular aspect, the medicament carrier is a blister pack comprising a base sheet and a lid (e.g. in strip form) and the coating is located in at least part of the sealed elements thereof such as the edge seal. Methods involving the coating (e.g. by plasma coating) of the edge seal(s) of a blister pack are particularly suitable. The coating may be applied before or after the blister pack is sealed (e.g. by heat-sealing). In aspects, coating post-sealing is preferable.

In another aspect, the coating material coats the wall, or the base sheet, or the lid of a medicament carrier in blister pack (e.g. blister strip) form.

In one aspect, the blister pack comprises a laminate comprising a coating material herein. Suitably, the laminate comprises material selected from the group consisting of metal foil, organic polymeric material and paper. Suitable metal foils include aluminium or tin foil having a thickness of from 5 to 100 μm, preferably from 10 to 50 μm, such as 20 to 30 μm. Suitable organic polymeric materials include polyethylene, polypropylene, polyvinyl chloride and polyethylene terephthalate.

Suitably, the base sheet and lid comprise different materials.

Suitably, the medicament container or delivery apparatus/device additionally comprises a desiccant material that is suitably either impregnated therein, or coated thereto. Suitably, the desiccant is selected from the group consisting of silica gel, zeolite, alumina, bauxite, anhydrous calcium sulphate, activated bentonite clay, water-absorbing clay, molecular sieve and any mixtures thereof.

In one aspect, there is also provided a method of reducing water ingress into a medicament powder comprising using a suitably modified medicament container or delivery apparatus/device herein,

In another aspect, there is also provided a package for storage of a medicament container or delivery apparatus/device herein formed from a material capable of controlling the ingress of moisture thereto or egress or moisture therefrom.

In one aspect, the moisture control material is impermeable to moisture.

In another aspect, the moisture control material controls the ingress or egress of moisture such that the ambient moisture content within the package is essentially constant, such as varying by no more than ±20%, preferably by less than ±10%. Ambient moisture content may for example be measured by Relative Humidity within the package. The preferred absolute level of moisture content will vary from medicament to medicament but may be readily determined through laboratory testing.

In another aspect, the moisture control material enables moisture transfer in one way only i.e. ingress only or egress only.

In another aspect, the moisture control material enables moisture transfer to either a set minimum/maximum moisture content within the package or within a set minimum/maximum moisture transfer rate.

In another aspect, the moisture control material is also capable of controlling the flow of other gaseous or vapour form species. Tyvek (trade name) is a suitable material.

In one aspect, the package is wrappable and sealable around the container to form an enclosed volume in which the container is disposed, the package being impermeable to water vapour, thereby substantially reducing ingress of water vapour and particulate matter into said enclosed volume.

In another aspect, the package additionally comprises a desiccant within the enclosed volume.

In one aspect the package includes at least one heat sealable layer and at least one layer of a metal foil. Suitably, said metal foil is selected from the group consisting of aluminium, tin, iron, zinc and magnesium.

In yet another aspect, the package includes protective layers located on the outside of the package. Suitably, the protective layer comprises a polyester film and the heat sealable layer comprises an ionomer film.

In a method aspect, there is provided a method of storing a medicament container or delivery apparatus/device herein comprising providing a packaging material which is capable of controlling the flow of water vapour; filling the medicament container or delivery apparatus/device with a medicament powder; wrapping said medicament container or delivery apparatus/device with said package material to form an enclosed volume in which said medicament container or delivery apparatus/device is disposed therein; and sealing the package.

In yet another aspect, the method additionally comprises providing a desiccant within the enclosed volume.

In one aspect, the sealing comprises heat-sealing said packaging material. In other aspects, the seal is formed by ultrasonic welding, heat stamping, adhesive or laser welding methods.

In another aspect of the present invention there is provided a packaged container, comprising a medicament container or delivery apparatus/device containing a medicament powder; and an overwrap package enclosing the medicament container or delivery apparatus/device and a desiccant; wherein the medicament container or delivery apparatus/device and the desiccant are sealable within the overwrap. Preferably the overwrap comprises a desiccant material and/or is lined, coated or impregnated with a desiccant material.

The overwrap package may be in the form of a shrink-wrap or of a loose wrap e.g. in sachet form. Any spare volume within the overwrap may be evacuated or an inert gas such as nitrogen deliberately inserted.

In another aspect, the medicament container or delivery apparatus/device medicament powder is formed from a material capable of controlling the ingress of moisture thereto or egress or moisture therefrom.

In one aspect, the material is impermeable to moisture. In another aspect, the material controls the ingress or egress of moisture such that the ambient moisture content within the package is essentially constant, such as varying by no more than ±20%, preferably by less than ±10%. In another aspect, the material enables moisture transfer in one way only i.e. ingress only or egress only. In another aspect, the material enables moisture transfer to either a set minimum/maximum moisture content within the package or within a set minimum/maximum moisture transfer rate.

In another aspect, the material is also capable of controlling the flow of other gaseous or vapour form species.

In other aspects, the medicament container or delivery apparatus/device or overwrap therefor is comprised of a material having desiccant blended or otherwise loaded or impregnated therein. Suitable materials are described in PCT Application Nos. WO99/62697 and WO/00/17258 in the name of Capitol Speciality Plastics Inc.

Suitable materials comprise a thermoplastic/desiccant blend. Examples of thermoplastics include polyolefin, polyethylene, polycarbonate, polyamide, ethylene-vinyl acetate copolymer, ethylene-methacrylate copolymer, polyvinyl chloride, polystyrene, polyester, polyester amide, polyacrylic ester, and polyvinylidene chloride, acrylic, polyurethane, polyacetal, and polycarbonate. These and other thermoplastics may be utilized either singularly, or in combinations.

The concentration of desiccant entrained (e.g. mixed or blended) within the thermoplastic may exceed seventy-five percent (75%) to not greater than eighty percent (80%) by weight, so that about seventy-five percent (75%) may extend to eighty percent (80%) by weight. Typically, however, the desiccant concentration will fall within a range of forty to seventy-five percent (40-75%) desiccant to thermoplastic, by weight. This concentration is considered to be a high concentration for most thermoplastics. The maximum desiccant bearable concentrations will vary among the various types of thermoplastics due to their differing characteristics. In the instance of polyethylene or polypropylene, for example, the maximum concentration of desiccant will be about seventy-five percent (75%) by weight. As the desiccant concentrations within the thermoplastics increase, the performance of the material degenerates to unacceptable levels. At lower levels of desiccant concentrations, about forty percent (40%) could extend to as low as thirty percent (30%) where the limits of a viable product are reached.

Suitable packaging material herein can be any material that is impervious to or substantially impervious to moisture. The packaging material is preferably permeable to volatiles which may escape from the plastics forming the body of the inhaler and/or the medicament carrier, by diffusion or otherwise, thereby preventing a build-up in pressure.

For ease of manufacturing, and in order to provide the desirable properties to the packaging material, the flexible packaging material preferably comprises a non-thermoplastic substrate (such as a metal foil) and a heat sealable layer disposed thereon, and an additional protective layer, such as a polymer film of polyester. The heat sealable layer is usually disposed on the inner surface of the assembled package. The additional protective layer is usually disposed on the surface opposite the heat sealable layer. An example of a particularly useful foil laminate is a polyester film adhesively laminated to aluminium foil adhesively laminated to lonomer (SURLYN™) film, for example, 12μ polyester/9μ aluminum/50μ ionomer film supplied by Lawson Mardon Singen (LMS). To further reduce moisture ingress, thicker metal films, such as 20 to 25μ, may be used.

The substrate is preferably formed from aluminium foil. However, other metals for the substrate include, but are not limited to, tin, iron, zinc, or magnesium formed on a sheet by vacuum deposition or sputtering and a carboxyl group-containing polyolefin layer formed on the metal layer by lamination.

The heat sealable layer can be formed from any thermoplastic or thermosetting material such as an ionomer resin, polyolefin, or cycloolefin copolymer. Ionomer resins typically include ionically cross-linked ethylene-methacrylic acid and ethylene acrylic acid copolymers. Properties which distinguish these ionomers resins from other polyolefin heat-sealed polymers are high clarity, high impact resistance, low haze in lamination, tear resistance, abrasion resistance, solid-state toughness, and moisture imperviousness. In the preferred embodiment, the heat sealable layer is made out of SURLYN™ (an ionomer resin) or a form of polyethylene to provide sufficient heat sealing properties.

The outer protective layer, if present, can be formed of any material as long as the final laminate has the requisite properties.

Preferably, the protective layer (e.g., polyester) is adhesively laminated to the substrate (e.g., aluminium) and the substrate layer in turn is adhesively laminated to the heat sealable layer (e.g., the ionomer film or SURLYN™ (an ionomer resin)). Preferred exemplary thicknesses of the three layers include a protective layer 1 to 40, preferably 4 to 30, more preferably 10 to 23 microns, and most preferably 12 microns; a substrate layer of 1 to 100, preferably 3 to 70, more preferably 5 to 50 microns, more preferably 6 to 20 microns, and most preferably 9 microns. For the heat sealable layer, preferred exemplary thicknesses include thicknesses of 1 to 100, preferably 5 to 70, more preferably 10 to 60, more preferably 20 to 55 microns, and most preferably 50 microns.

Adhesives may be used to join the respective layers of materials together. The adhesive layers are typically substantially smaller in thickness relative to the thickness of the substrate, heat sealable and/or protective layers which they bond. The number, size, and shape of the layers are not limited to those layers shown in the drawings. Any number of layers with relative areas of any size and predetermined thicknesses may be used so long as the flexible package forms an enclosed volume which substantially prevents ingression of water vapour and particulate matter into the enclosed volume while permitting egression out of the enclosed volume of any volatile released from the plastics used in the body of the inhaler or the medicament carrier. The size, shape, and number of layers of the package are typically a function of the size and contents of the inhaler and/or medicament carrier.

The package is believed to operate similarly to a virtual one-way valve due to the composition of the layers and due to the transmission rate of water vapour molecules into the enclosed volume relative to the transmission rate of gas molecules of a plastic volatile, such as formaldehyde, out of the enclosed volume. The package permits the volatile to diffuse out of the enclosed volume while substantially preventing water vapour and other particulate matter from entering the enclosed volume. Excess or leakage of the volatile is permitted to egress from the package. The virtual one-way valve function of the package prevents or minimizes the chance of any sudden ruptures or prevents or minimizes unexpected expulsion of the plastic volatile during opening of the package.

Suitable vapour or moisture absorbing materials include desiccants made from inorganic materials such a zeolites and aluminas. Such inorganic materials of vapour or moisture absorbing materials have high water absorption capacities and favourable water absorption isotherm shapes. The water absorption capacity of such materials typically varies from 20 to 50 weight percent.

In the preferred embodiment, the absorbing material is a MlNIPAX® supplied by Multisorb Technologies in the United States and Silgelac in Europe (silica gel packaged inside TYVEK®, which is a nylon mesh bonded with a microporous polyurethane). Other exemplary moisture absorbing materials include, but are not limited to, alumina, bauxite, anhydrous, calcium sulphate, water-absorbing clay, activated bentonite clay, a molecular sieve, or other like materials which optionally include a moisture sensitive colour indicator such as cobalt chloride to indicate when the desiccant is no longer operable. Whilst the package is suitably designed to substantially prevent ingression of water vapour and particulate matter into the enclosed volume, the moisture absorbing material is placed within the enclosed volume in order to absorb any residual moisture present in the atmosphere or on the external surface of medicament container or delivery device/apparatus or a combination thereof, prior to sealing the package.

The desiccant may be present in an amount sufficient to absorb any residual moisture inside the package. When silica gel is used, 1 g to 10 g of silica gel is sufficient for a typical dry powder inhaler. Moreover, the desiccant may be present in an amount sufficient to absorb any moisture that possibly ingresses from the external environment. It is also possible to place the desiccant inside the container, either loose therein or as part of an assembly attached to the canister.

### BRIEF DESCRIPTION OF THE DRAWINGS

FIG. 1 shows a medicament carrier in the form of a capsule according to the present invention.

FIG. 2a is a cross-sectional side elevation of a single medicament blister strip according to the present invention.

FIG. 2b is a top perspective of a medicament blister strip illustrated in FIG. 2a.

FIG. 3a is a cross-sectional side elevation of a single medicament blister having a laminate according to the present invention.

FIG. 3b is a top perspective of a medicament blister strip illustrated in FIG. 3a.

FIG. 4a is a cross-sectional side elevation of a single medicament blister having a ring surrounding the blister pocket.

FIG. 4b is a top perspective of the medicament blister shown in FIG. 4a.

FIG. 5 shows a cross-sectional dry powder inhaler comprising a powder reservoir according the present invention.

FIG. 6 shows a cross-sectional dry powder inhaler comprising a medicament carrier according to the present invention.

FIG. 7 is a top perspective of a package for storing a dry powder inhaler according to the present invention.

FIG. 8 is a side perspective of the package of FIG. 7.

FIG. 9 is a cut-away bottom perspective of the package for storing a dry powder inhaler according to the present invention.

FIG. 10 is a cross-sectional view of the package for storing a dry powder inhaler according to the present invention.

### DETAILED DESCRIPTION OF THE DRAWINGS

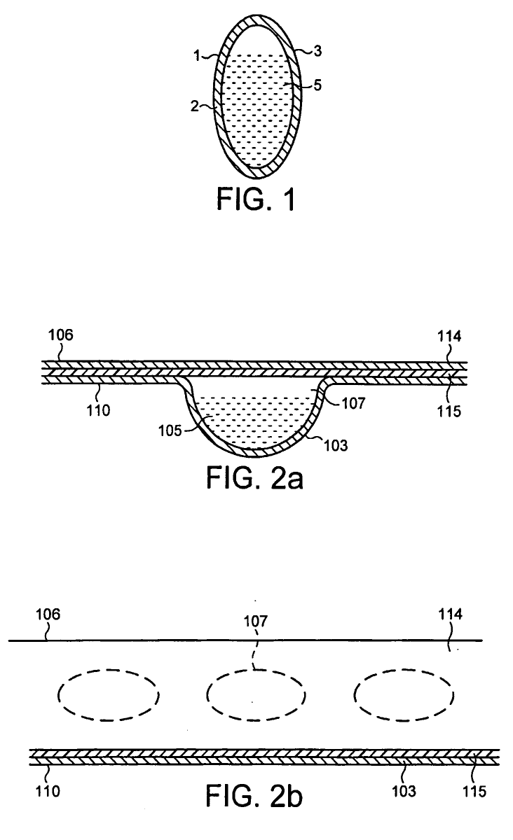

The medicament carrier in FIG. 1 is in the form of a capsule 1 comprising a wall 2 enclosing medicament powder 5. The wall 2 is plasma coated with a fluoropolymer coating 3 which reduces the surface energy of the capsule 1. Medicament powder 5 is released on piercing the wall 2 of capsule 1 and may be inhaled by a patient.

FIG. 2a shows a sectional side-elevation of a single blister strip 106 comprising a pocket 107, containing dry powder 105, base 110 and lid comprising laminates 114, 115. The lid is composed of a metallic foil laminate 114 bound to a plastic laminate 115. In the diagram, the lid 114, 115 is hermetically sealed to base 110 by appropriate means (e.g. adhesion, welding). Base 110 is plasma coated with a fluoropolymer coating 103. In use, the fluoropolymer coating maintains the powder 105 in a flowable condition until the lid 114, 115 is removed from the base 110.

A top perspective of the blister strip 106 showing pockets 107 is illustrated in FIG. 2b. Laminated lid 114, 115 is sealed to base 110 which is plasma coated with fluoropolymer coating 103.

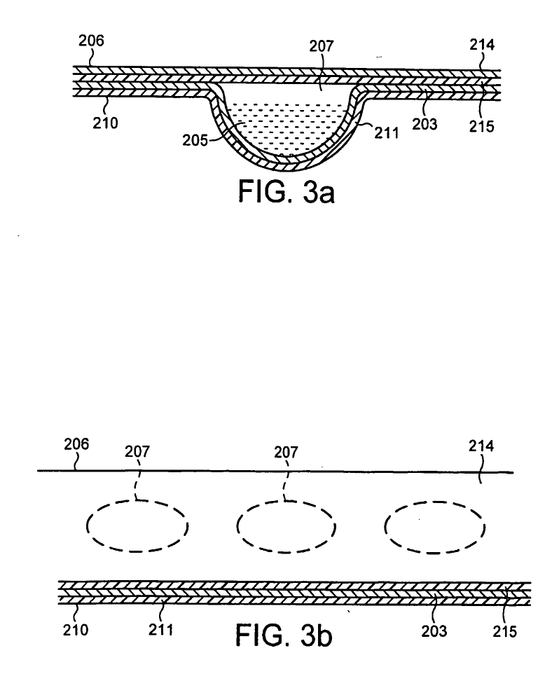

FIG. 3a shows a cross-sectional elevation of a different single blister strip 206 according to the invention. The blister strip 206 is composed of several laminated sheets, the lid being formed from metallic foil 214 and plastic laminate 215 while the base comprises plastic laminates 210 and 211. The plastic laminate 211 comprises a plasma fluropolymer coating 203 for absorbing any moisture which permeates through laminated sheets 214, 215 and 210, thereby reducing ingress into medicament powder 205 within pocket 207.

FIG. 3b is a top perspective of a blister strip 206 showing several blisters as described in FIG. 3a. Metallic foil 214 and plastic laminate 215 form a lid which is hermetically sealed, by appropriate adhesive or welding means, to the base of strip 206. The base comprises plastic laminates 210 and 211, laminate 211 being disposed on the internal surface of pocket 207 and comprising a plasma fluropolymer coating.

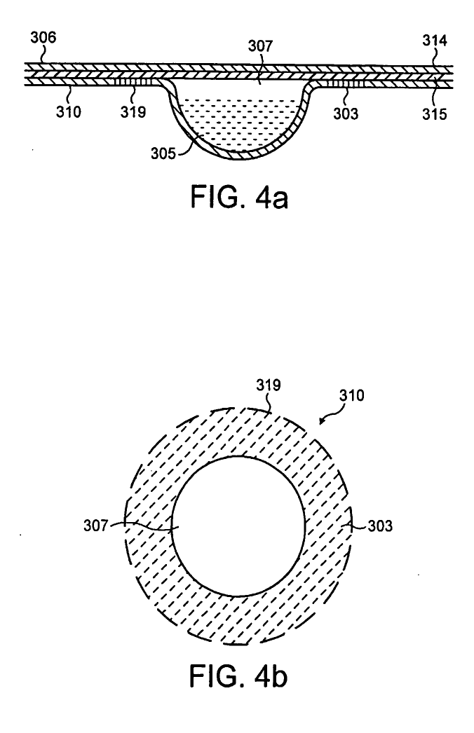

FIG. 4a shows a cross-sectional elevation of yet another plasma fluoropolymer coated single blister strip 306 according to the invention. Pre-plasma coated metallic foil 314 and plastic laminate 315 form a lid for base 310 which are hermetically sealed together to reduce moisture ingress into pocket 307 containing medicament powder 305. The circumference of pocket 307 is surrounded by a ring 319, within base 310, comprising desiccant 303 which absorbs moisture which permeates into the blister, particularly between lid sheet 315 and base sheet 310.

A plan perspective of the single blister strip 314 shown in FIG. 4a is illustrated in FIG. 4b. The ring 319 of material comprising desiccant 303 surrounds pocket 307 thereby absorbing any moisture which permeates into the pocket 307 through foil 314 and laminates 315 and/or base sheet 310, together with moisture ingress between lid and base sheets 315, 310.

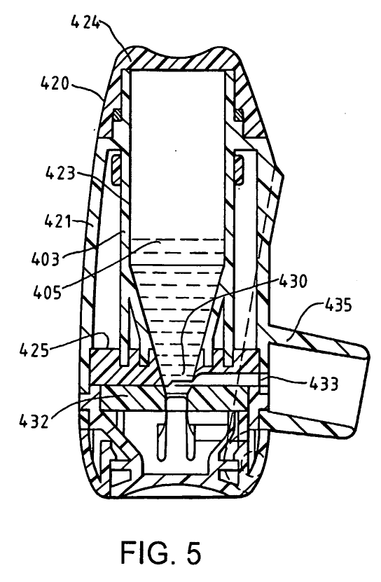

FIG. 5 shows a sectional view of a dry powder inhaler 420 according to the present invention. The inhaler 420 comprises a body 421 which defines a reservoir 423 and a reservoir cover 424. The reservoir contains a supply of medicament in dry powder form 405. The walls 423 of the reservoir, defined by the body 421, are plasma coated with a fluoropolymer coating 403. Base 425 and body 421 define an aperture 430 through which powder 405 can pass from the reservoir to the dosing member 432. Powder 405 is guided by the walls 423 of the reservoir, which form a hopper, to the dosing member 432. Extending laterally from the lower end of the main body 421 is mouthpiece 435, through which the patient inhales via passage 433. If the device were intended for nasal inhalation this would be replaced by a nosepiece. The fluoropolymer material 403 comprising walls 423, reduce adherence by medicament powder 405. Optionally a desiccant comprising material may be located within the walls of passage 433 and/or a ring of same material around the metering valve (not shown) which controls the flow of medicament into passage 433.

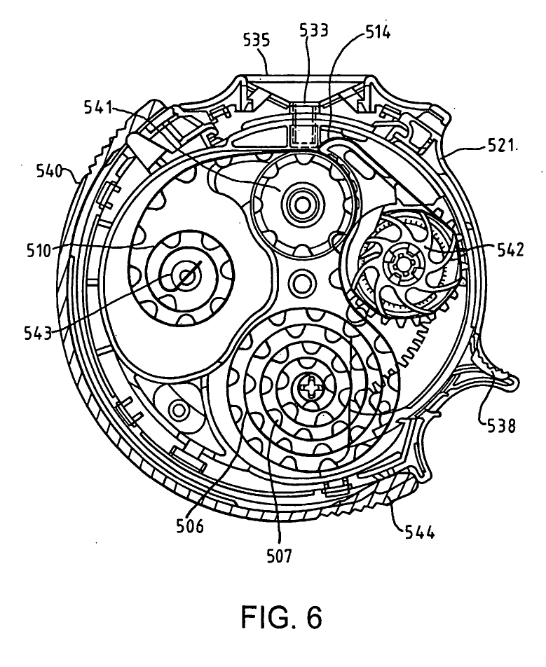

FIG. 6 shows a simplified cross-sectional plan view of a dry powder inhaler comprising a medicament carrier according to the present invention. The inhaler 540 dispenses unit doses of medicament powder from a medicament blister strip 506. The inhaler is comprised of an outer casing 544 enclosing a fluoropolymer plasma coated medicament strip 506 within body 521. The medicament strip may be, for example, any of those described in FIGS. 2a to 4b above. The internal walls of body 521 are coated with a desiccant material to reduce the moisture content of the interior of the inhaler. The patient uses the inhaler by holding the device to his mouth, depressing lever 538, and inhaling through mouthpiece 535. Depression of lever 538 activates the internal mechanism of the inhaler, such that the lid 514 and base 510 sheets of coiled medicament blister strip 506 are separated at index wheel 541 by use of contracting wheel 542 and base wheel 543. A unit dose of powdered medicament within blister pocket 507 is released and may be inhaled by the patient through exit port 533 and mouthpiece 535.

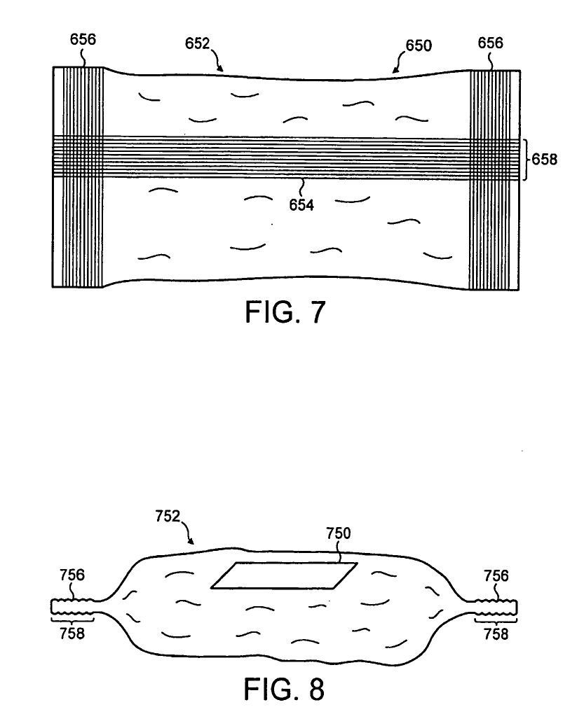

FIG. 7 shows a top perspective of a container storage system for storing a dry powder inhaler or cartridge refill therefor according to the present invention. The container storage system 650 includes a package or wrapping 652 that employs multi-layers of material 970, 972, 974. (See FIG. 10.) The package 652 further includes fin seams 654, 656 which are disposed along two parallel side edges of the package and along a single longitudinal edge of the package 652. The package 652 comprises a desiccant material, or alternatively is lined, coated or impregnated with a desiccant material.

The number and type of fin seams 654, 656 are not limited to the types shown in the drawings. The package 652 can include additional seams or significantly fewer seams such as a continuous single seam. The orientation of the seams 654, 656 is not limited to the orientation shown in the drawings. The orientation of the seams 654, 656 is typically a function of the sealing device and such seams may be oriented in a manner which substantially increases manufacturing efficiency. During manufacture, the longitudinal seam 654 may be formed first by heat sealing and the two end seams 656 may then be formed by heat sealing to close the package. Other types of seams include, but are not limited to, gusset type seams which include excess material which provides expansibility, stitched type seams, or mechanically crimped seams, and other like structures.

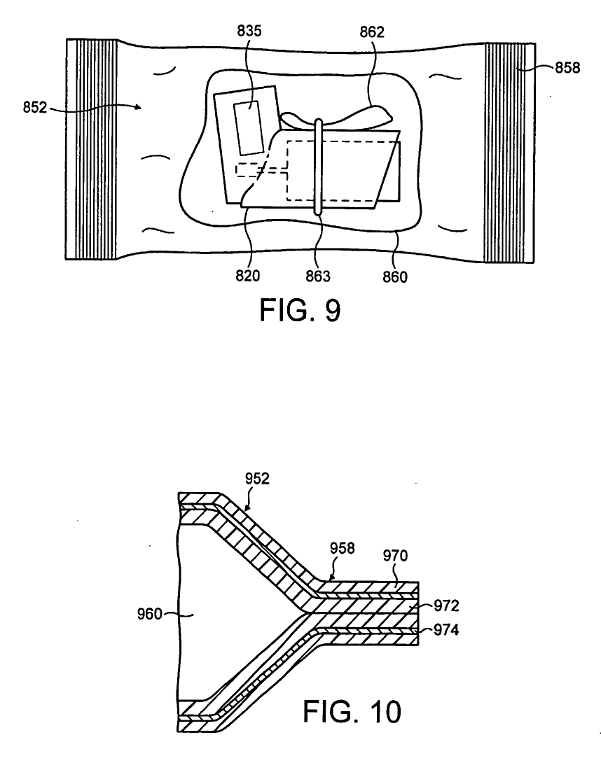

The container storage system includes a dry powder inhaler 820 (see FIG. 9). While the preferred inhaler is a dry powder inhaler 820, other dry powder inhalers (such as that described in FIG. 6) are not beyond the scope of the present invention.

FIG. 8 shows a side perspective of the container storage system of FIG. 7. The fin seams 654 and 656 in FIG. 7 are formed by a conventional heat sealing device which mechanically crimps sides of the package 750 together while simultaneously providing heat to the sides 654, 656/756 (FIGS. 7 and 8). The heat sealing device typically has electrical heater elements shaped to produce the pattern of the fin seams 654, 656/756 where the fin seams include multiple ridges 658/758. The sealing mechanism of the container storage system 6501750 of the present invention is not limited to heat sealing devices. Other sealing devices include, but are not limited to, glue sealing machines, sonic welding machines, electron beam radiation machines, and other like sealing devices.

As shown in FIG. 7, the package 750 preferably has a substantially rectangular configuration with a substantially elliptical cross section, however, other shapes of the package 750 are not beyond the scope of the present invention. Other shapes include, but are not limited to circular, square, triangular, trapezoidal, pentagonal, hexagonal, octagonal, and other like shapes. The shape of the package 750 is preferably a function of the shape of the enclosed medicament powder container 34 as well as the amount and type of storage space since the package 752 is made from flexible materials as will be described in further detail below.

FIG. 9 shows a cut-away bottom perspective of the package for storing a dry powder inhaler according to the present invention. The package 852 provides an enclosed volume 860 in which the inhaler 820 is disposed therein. The size of the enclosed volume 860 can be adjusted according to the size of the inhaler 820 and related parts thereto. Preferably, the enclosed volume 860 is of a size which permits relative ease of closing respective sides and layers 852, 26 and 28 without substantial stretching of the package 852. The enclosed volume 860 may be substantially evacuated prior to formation of the fin seams 858, 854 (not shown) to substantially reduce any water vapour being present in the enclosed volume 860. The enclosed volume 860 may be evacuated to such a degree that the enclosed volume 860 is a vacuum region around the medicament inhaler 820. While the enclosed volume 860, may remain constant, its relative shape may change according to shifting of the inhaler 820 disposed within the enclosed volume 860.

In a preferred embodiment, a porous container of moisture absorbing material 862 lays adjacent to the mouthpiece 835 in a loose or free flowing manner. Alternatively, the moisture absorbing material 862 can be secured to the inside of the flexible package. In another alternative embodiment, the moisture absorbing container 862 may be attached to a bracket structure such as a ring which is fastened to the inhaler 820.

In one possible embodiment, the moisture absorbing material may be attached to the external surface of the mouthpiece 835 by a fastening device such as a rubber band 863. The fastening device 863 is preferably a removable elastic mechanism such as a rubber band. However, other fastening devices are not beyond the scope of the present invention. Other fastening devices include, but are not limited to, adhesives, adhesive tapes, shrink-wrap plastic, fasteners such as screws, nails, or rivets, compartments which are part of the mouthpiece housing 46, and other like attachment devices. In an alternative embodiment (not shown), a plurality of beads of material comprising a desiccant may be placed within the enclosed space 860. Similarly, other carriers comprised of a desiccant material may be enclosed within space 860 to absorb excess moisture from the enclosure.

FIG. 10 is a cross-sectional view of the package for storing a dry powder inhaler according to the present invention. The amorphous shape of the enclosed volume 960 is attributed to the flexible materials which make up the layers 970, 972, 974 of the package 952. The enclosed volume 960 can be varied in size such that it substantially conforms to the shape of the inhaler and any related parts thereto or such that the enclosed volume 960 is larger than the inhaler 820, as shown in FIG. 9. When the enclosed volume is of a size which is substantially equivalent with the surface area of the inhaler 820 and related parts, the layers 970, 972, and 974 of material substantially conform to the shape of the inhaler and related parts. The package is preferably placed in a separate, more rigid container, such as a paperboard or cardboard box (not shown) typically used in the pharmaceutical industry.

Administration of medicament may be indicated for the treatment of mild, moderate or severe acute or chronic symptoms or for prophylactic treatment. It will be appreciated that the precise dose administered will depend on the age and condition of the patient, the particular medicament used and the frequency of administration and will ultimately be at the discretion of the attendant physician. When combinations of medicaments are employed the dose of each component of the combination will in general be that employed for each component when used alone. Typically, administration may be one or more times, for example from 1 to 8 times per day, giving for example 1,2,3 or 4 puffs each time. Each dose, for example, may deliver 5 μg, 50 μg, 100 μg, 200 μg or 250 μg of a medicament. Typical dry powder inhalers comprise 100, 120 or 200 doses of medicament; the dosage of each medicament is either known or readily ascertainable by those skilled in the art.

Appropriate medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone e.g. as the furoate ester), ciclesonide, triamcinolone (e.g. as the acetonide) or 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); α₄ integrin inhibitors e.g. (2S)-3-[4-({[4-(aminocarbonyl)-1-piperidinyl]carbonyl}oxy)phenyl]-2-[((2S)-4-methyl-2-[2-(2-methylphenoxy)acetyl]amino}pentanoyl)amino]propanoic acid (e.g. as free acid or potassium salt), diuretics, e.g., amiloride; anticholinergics, e.g., ipratropium (e.g. as bromide), tiotropium,; atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

Preferred respiratory medicaments are selected from albuterol, salmeterol, fluticasone propionate and beclomethasone dipropionate and salts or solvates thereof, e.g., the sulphate of albuterol and the xinafoate of salmeterol.

Preferred medicament combination products contain salbutamol (e.g., as the free base or the sulphate salt) or salmeterol (e.g., as the xinafoate salt) or formoterol (eg as the fumarate salt) in combination with an anti-inflammatory steroid such as a beclomethasone ester (e.g., the dipropionate) or a fluticasone ester (e.g., the propionate) or budesonide. A particularly preferred combination of components comprises fluticasone propionate and salmeterol, or a salt thereof (particularly the xinafoate salt). A further combination of components of particular interest is budesonide and formoterol (e.g. as the fumarate salt).

Generally, powdered medicament particles suitable for delivery to the bronchial or alveolar region of the lung have an aerodynamic diameter of less than 10 micrometers, preferably less than 6 micrometers. Other sized particles may be used if delivery to other portions of the respiratory tract is desired, such as the nasal cavity, mouth or throat. The medicament may be delivered as pure drug, but more appropriately, it is preferred that medicaments are delivered together with excipients (carriers) which are suitable for inhalation. Suitable excipients include organic excipients such as polysaccharides (i.e. starch, cellulose and the like), lactose, glucose, mannitol, amino acids, and maltodextrins, and inorganic excipients such as calcium carbonate or sodium chloride. Lactose is a preferred excipient.

Particles of powdered medicament and/or excipient may be produced by conventional techniques, for example by micronisation, milling or sieving. Additionally, medicament and/or excipient powders may be engineered with particular densities, size ranges, or characteristics. Particles may comprise active agents, surfactants, wall forming materials, or other components considered desirable by those of ordinary skill.

The excipient may be included with the medicament via well-known methods, such as by admixing, co-precipitating and the like. Blends of excipients and drugs are typically formulated to allow the precise metering and dispersion of the blend into doses. A standard blend, for example, contains 13000 micrograms lactose mixed with 50 micrograms drug, yielding an excipient to drug ratio of 260:1. Dosage blends with excipient to drug ratios of from 100:1 to 1:1 may be used. At very low ratios of excipient to drug, however, the drug dose reproducibility may become more variable.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein: They may take the form of product, method or use claims and may include, by way of example and without limitation, one or more of the following claims:

1. A medicament container containing medicament in dry powder form, said medicament container comprising a material that reduces the surface energy or increases the electrical conductivity of some or all parts of the medicament container which come into contact with said dry powder form medicament, wherein the medicament container is in the form of a capsule or blister pack and with the proviso that said material is free from desiccant.

2. A medicament container according to claim 1, wherein said material coats said some or all parts of the medicament container which come into contact with said dry powder form medicament.

3. A medicament container according to claim 1, wherein said material incorporates into the some or all parts of the medicament container which come into contact with said dry powder form medicament.

4. A medicament container according to claim 1, wherein the material is a friction-reducing material that acts such as to reduce the surface energy of the some or all parts of the medicament container which come into contact with the dry powder form medicament.

5. A medicament container according to claim 4, wherein said surface energy is reduced such as to give a contact angle of greater than about 70 degrees, preferably greater than about 100 degrees.

6. A medicament container according to claim 1, wherein the material comprises a fluoropolymer.

7. A medicament container according to claim 6, wherein said fluoropolymer is selected from the group consisting of tetrafluoroethylene (PTFE), fluorinated ethylene propylene (FEP), perfluoroalkoxyalkane (PFA), ethylene tetrafluoroethylene (ETFE), vinyldienefluoride (PVDF), chlorinated ethylene tetrafluoroethylene and any mixtures thereof.

8. A medicament container according to claim 7, wherein the fluoropolymer is blended with a non-fluorinated polymer selected from the group consisting of polyamides, polyimides, polyamide imides, polyethersulfones, polyphenylene sulfides, and amine-formaldehyde thermosetting resins.

9. A medicament container according to claim 6, wherein the fluoropolymer is applied by surface treatment of the medicament container.

10. A medicament container according to claim 9, wherein said surface treatment comprises a process of plasma coating.

11. A medicament container according to claim 10, wherein said process of plasma coating employs fluorinated small molecules selected from the group consisting of C₁₋₁₀perfluoroalkanes, C₂₋₁₀perfluoroalkenes and any mixtures thereof.

12. A medicament container according to claim 10, wherein said process of plasma coating is by interchange of hydrogen ions at the surface of the container or delivery device with fluorine ions.

13. A medicament container according to claim 10, wherein the plasma coating process is selected from the group consisting of a microwave plasma process, a pulsed wave plasma process and a hot filament chemical vapour process.

14. A medicament container according to claim 1, wherein said material is a conductivity-enhancing material that acts such as to enhance the electrical conductivity of the some or all parts of the medicament container which come into contact with the dry powder form medicament.

15. A medicament container according to claim 14, wherein the conductivity-enhancing material is a ceramic material.

16. A medicament container according to claim 15, wherein said ceramic material comprises a composite of a ceramic material with a conductive metal or a ceramic material doped with a conductive metal.

17. A medicament container according to claim 15, wherein the conductivity-enhancing material comprises an organic polymeric material.

18. A medicament container according to claim 17, wherein said organic polymeric material is selected from the group consisting of polycarbonate, polyester, acetal, nylon and epoxy resins.

19 A medicament container according to claim 17, wherein the organic polymeric material includes a doping element selected from the group consisting of gold, silver and similar precious metals, carbon black and quaternary ammonium salts.

20. A medicament container according to claim 1, wherein the dry powder medicament is suitable for the treatment of a respiratory disorder.

21. A medicament container according to claim 20, wherein said dry powder medicament is selected from the group consisting of albuterol, salmeterol, fluticasone propionate and beclomethasone dipropionate and salts or solvates thereof and any mixtures thereof.

22. A medicament container according to claim 21, wherein the dry powder medicament comprises a combination of fluticasone propionate and salmeterol xinafoate.

23. An inhalation device comprising a body defining a chamber for receipt of a dry powder medicament container according to claim 1 and an outlet in communication with said chamber for release of the medicament.
